(19) United States
(12) Patent Application Publication
(10) Pub. No.: US 2024/0293014 A1
(43) Pub. Date: Sep. 5, 2024

(54) ENDOSCOPE SYSTEM

(71) Applicant: Richard Wolf GmbH, Knittlingen (DE)

(72) Inventors: Frederick POTHOF, Bruchsal (DE); Dirk GÖTHEL, Kürnbach (DE); Sebastian FREY, Waghäusel (DE)

(21) Appl. No.: 18/572,865

(22) PCT Filed: Jun. 20, 2022
(86) PCT No.: PCT/DE2022/200135

§ 371 Date: Dec. 21, 2023

(30) Foreign Application Priority Data
Jun. 25, 2021 (DE) 10 2021 206 584.8

(51) Int. Cl. A61B 1/015; A61B 1/018; A61B 1/06

(52) U.S. Cl. CPC A61B 1/015; A61B 1/018; A61B 1/0676; A61B 1/0684; A61B 2217/007

(57) ABSTRACT

An endoscope system includes an endoscope (4) inserted into a working sleeve (2). A first fluid channel (20) is open to the distal end and is formed in the inside of the endoscope. An outer cross section of the endoscope and the inner cross section of the working sleeve are matched to one another. With the endoscope inserted into the working sleeve, in a first section (14) of its outer periphery, it bears on the inner periphery of the working sleeve. A second section (16) of its outer periphery is distanced to the inner periphery of the working sleeve. A free space (18) extends over an axial length (X) of the working sleeve and forms a second fluid channel which is open to the distal end and is formed between the inner periphery of the working sleeve and the second section of the outer periphery of the endoscope.

### CROSS REFERENCE TO RELATED APPLICATIONS

This application is a United States National Phase Application of International Application PCT/DE2022/200135, filed Jun. 20, 2022, and claims the benefit of priority under 35 U.S.C. § 119 of German Application 10 2021 206 584.8, filed Jun. 25, 2021, the entire contents of which are incorporated herein by reference.

### TECHNICAL FIELD

The invention relates to an endoscope system, in particular to an endoscope system for spinal column surgery.

### BACKGROUND

Operations, in particular fully-endoscopic operations of the spinal column are often carried out amid continuous fluid rinsing. The continuous rinsing of fluid ensures a free field of view, by way of blood and tissue particles being transported away out of the field of view by the rinsing. Apart from the reliable rinsing, there is also the difficultly that the intra-operative pressure should not rise excessively during the spinal column surgery.

### SUMMARY

It is an object of the invention to provide an improved endoscope system, in particular an endoscope system for spinal column surgery, which permits an improved rinsing of the operational region whilst avoiding an undesirable pressure increase.

This object is achieved by an endoscope system with features which are specified herein. Preferred embodiments result from the subsequent description as well as the attached figures.

The endoscope system according to the invention as essential elements comprises a working sleeve as well as an endoscope which can be inserted into the working sleeve. The endoscope can be pushed into the working sleeve from the distal end. Herein, the endoscope is preferably displaceable in the working sleeve in the axial direction, as well as rotatable in its angular position about a longitudinal axis. The endoscope as well as the working sleeve are preferably configured as rigid elements or shanks. A first fluid channel which is open to the distal end is formed in the inside of the endoscope. This means that the fluid channel has an opening at the distal end of the endoscope. Moreover, the endoscope at its proximal end comprises a fluid connection, at which the fluid channel can be connected to a supply device, in order to lead fluid through the fluid channel. A shut-off cock and for example a suitable connection element for the connection to a hose can be arranged on the fluid connection in a known manner.

The outer cross section of the endoscope and the inner cross section of the working sleeve, according to the invention are specially matched to one another in a manner such that the endoscope when it is inserted into the working sleeve, in a first section of its outer periphery bears in the inner periphery of the working sleeve and in a second section of its outer periphery is distanced to the inner periphery of the working sleeve. Herein, the cross section relates to a plane normal to the longitudinal axis of the working sleeve which extends from the proximal to the distal end. In a first section, the outer periphery of the endoscope is configured corresponding to the inner contour of the working sleeve, so that preferably an extensive contact or parallel course of the walls is achieved. The first section preferably extends over more than 90 degrees, further preferably over at least half the inner periphery of the working sleeve, so that the endoscope preferably at least in a peripheral section of larger than 90 degrees and further preferably at least 180 degrees bears extensively on the inner periphery of the working sleeve or extends parallel to this. Herein, the contact region is preferably not interrupted in the circumferential direction. By way of this, in particular a guide for rotating the endoscope in the inside of the working sleeve can be realized. By way of the distancing between the outer periphery of the endoscope and the inner periphery of the working sleeve, a free space which created in the region between the inner periphery of the working sleeve and the second section of the outer periphery. This free space forms a second fluid channel which is open to the distal end. For this, the free space preferably extends over the whole axial length of the working sleeve, which is to say from the distal to the proximal end. Preferably, the cross section of the working sleeve and of the endoscope is constant over the axial extension from the proximal to the distal end. The working sleeve according to a preferred design comprises a second fluid connection in the region of its proximal end, said second fluid connection being in connection with the free space which defines the second fluid channel. This fluid connection can be a connection element for the connection to a hose, and possibly in the known manner a shut-off cock. A circulation of fluid can be effected through the first and the second fluid channel, wherein the fluid flows through the fluid channel in the inside of the endoscope in one direction and through the free space between the endoscope and the working sleeve in the other direction. Herein, the first fluid channel forms the feed channel, through which the fluid is led into the operational field, and the second fluid channel forms the return flow or discharge channel, through which the fluid and possibly particles or substances which are to be rinsed away are led away out of the operational field. Preferably, the endoscope with its outer periphery bears on the inner periphery of the working sleeve such that seen in the diameter direction, a free space is only formed at one side, i.e. at only one side of the instrument longitudinal axis, whereas the outer periphery of the endoscope bears on the inner periphery of the working sleeve at the side which is diametrically opposite to the free space. By way of this, a greater mechanical stability, in particular a greater resistance to buckling loads is created. The outer periphery of the endoscope bears on the inner periphery of the working sleeve over a continuous region of 180 degrees or more.

According to an embodiment, the endoscope at its distal end comprises a viewing window, for example in the form of an objective lens. The viewing window preferably has a circular cross section. The view into the operational field is effected through this viewing window or through this objective lens, wherein the image transmission can be effected by a video system or connecting endoscope optics. The viewing window is preferably arranged in the distal end surface of the endoscope in a sealed manner. The viewing window with the endoscope optics which connect thereto can herein define a straight viewing direction parallel to the instrument longitudinal axis or also an angled viewing direction. An angled viewing direction is preferably directed in the direction of the axial extension of a working channel, so that a working space which is situated distally in front of the working channel can be observed through the viewing window by way of the endoscope optics.

Preferably, an opening of the first fluid channel and the free space which forms the second fluid channel are situated at sides of the viewing window which are away from one another, further preferably sides which are essentially diametrically away from one another. By way of such a design, one succeeds in the fluid flowing from the one fluid channel into the other, preferably from the first fluid channel into the second fluid channel, via the viewing window, which means along the outer side of the viewing window. The field of view is thus continuously rinsed and a free view into the operation region is ensured.

Preferably, at the distal end, an opening of the first fluid channel and the free space which defines the second fluid channel are arranged relative to a viewing window, i.e. preferably to the viewing window of endoscope optics, as has been described beforehand, in a manner such that a fluid flow through a viewing field which is situated in front of the viewing window runs between the first and the second fluid channel. I.e. when fluid is fed through the first fluid channel and is led away through the second fluid channel, the fluid flow exits out of the first fluid channel at the distal end, runs through the viewing field in front of the viewing window and is led away through the second fluid channel. Alternatively, the fluid flow could also run in the reverse direction. If the viewing direction through the viewing window runs at an angle to the longitudinal axis of the instrument, then the opening of the first fluid channel and the free space are preferably situated such that the flow runs through the angled field of view field. For this, the opening and the free space can possibly be arranged offset in the viewing direction, preferably towards a working channel.

The working sleeve preferably comprises a circular inner cross section. Further preferably, the working sleeve as a whole is configured as a tube with a circular cross section. The circular inner cross section permits the rotation of the endoscope in the inside of the working sleeve, wherein the endoscope in the region of the first section of its outer periphery can slide along on the inner periphery of the working sleeve or move parallel to the inner periphery.

According to a further embodiment, the endoscope in the first section of its outer periphery has an outer contour which is arc-shaped on cross section, wherein further preferably the arc-shaped outer contour has a radius of curvature which corresponds essentially to the radius of the inner periphery of the working sleeve. The radii of the inner periphery of the working sleeve and of the outer contour of the endoscope can herein slightly differ from one another, so that a press fit is created which permits the arc-shaped outer peripheral section of the endoscope to slide along the inner periphery of the working sleeve or to move in parallel. The circular inner contour of the working sleeve herein extends concentrically to the longitudinal axis of the working sleeve which runs from the proximal end to the distal end. The arc-shaped outer peripheral section of the endoscope in the first section of the outer periphery of the endoscope herein curves about the same longitudinal axis which is herewith preferably also a rotation axis, about which the endoscope is rotatable in the inside of the working sleeve.

In the region of the second section of the outer periphery of the endoscope, its outer contour is preferably flattened or configured in the shape of an oval arc. This means that in this section for example the radius of the outer periphery is shorter than the radius of the inner periphery of the working sleeve, so that the free space which is described above is created.

As already described, the endoscope is preferably rotatable in the inside of the working sleeve about a longitudinal axis, preferably the longitudinal axis of the working sleeve. For this, the endoscope can be rotatably mounted or held in a receiver at the proximal end of the working sleeve. On rotation, as described, an outer peripheral surface of the endoscope slides along the inner wall of the working sleeve in the region of the first section. The free space which is formed between the second section of the outer periphery of the endoscope and the inner wall of the working sleeve and which functions as a second fluid channel herein rotates together with the endoscope about the longitudinal axis of the working sleeve. Inasmuch as this is concerned, the second fluid channel always retains a defined relative position to the endoscope, independently of its angular position in the inside of the working sleeve.

The second fluid channel preferably has a larger cross-sectional area than the first fluid channel. In particular, this is advantageous if the second fluid channel serves as a discharge or suction channel. If the return has a larger cross section than the feed through the first fluid channel, it is then ensured that no undesirable pressure increase forms in the operational region due to the fluid. Preferably, the free space has a cross section which is larger than the cross section of the first fluid channel by at least 20%, further preferably by more than 50%. The cross section of the free space can also be three to five times as large as the cross section of the first fluid channel or be configured larger.

The endoscope further preferably in its inside comprises a working channel which is opened towards the distal end, with a preferably circular cross section. The working channel extends parallel to the longitudinal axis of the endoscope and herewith preferably parallel to the longitudinal axis of the working sleeve. The working channel extends from the proximal to the distal end of the endoscope. The working channel is connected to a receiver for a working insert at the proximal end, wherein the receiver is preferably configured such that a working insert or instrument can be pushed through the receiver into the working channel in a sealed manner. Herein, the working instrument is preferably movable in the working channel in the known manner. In cross section, the working channel preferably lies next to the first fluid channel and possibly endoscope optics in the endoscope. The opening of the working channel at the distal end of the endoscope is preferably situated relative to the viewing window in a manner such that an instrument which is advanced through the working channel into the endoscope region is situated in the field of view through the viewing window.

According to a further preferred embodiment, a cross-sectional axis of the working channel, preferably a diameter axis of the working channel and a cross-sectional axis of the viewing window, preferably a diameter axis of the viewing window lie on a common straight line, wherein this common straight line further preferably runs parallel to or along the diameter of the working sleeve. This permits the optimal utilization of the diameter of the endoscope or of the working sleeve, in order to realize a working channel and a viewing window of maximal size. The straight line which is defined by the mentioned cross-sectional axes and the outer contour of in the first section of the outer periphery of the endoscope further preferably define a semicircle. This means that seen in cross section, the endoscope in its first section has a semicircular shape. In the second section however, the endoscope is configured in a flattened manner or has an oval outer contour which is smaller than the inner contour of the working sleeve, so that in the described manner a free space which is to say in particular an arc-shaped free space is formed between the endoscope and the inner periphery of the working sleeve and which in the described manner serves as a second fluid channel.

Expediently, the sum of the diameter of the viewing window and working channel is larger than 75%of the inner diameter of the working sleeve. Hence the inner cross section of the working sleeve is optimally utilized.

As described, the image transmission in the endoscope can be effected via a video system or endoscope optics. Concerning a video system, a video sensor could be arranged in the region of the distal end of the endoscope, which is to say in particular behind the viewing window or an objective which connects onto the viewing window. Alternatively, the picture can be transmitted from the distal end to the proximal end of the endoscope via rigid endoscope optics, for example a lens or fiber optic arrangement, wherein possibly a camera could be arranged in the region of the proximal end of the endoscope, e.g. on a handle.

Further preferably, the endoscope comprises an illumination device. For this, at least one light exit surface, via which the field of operation can be illuminated, is preferably arranged at the distal end. At least one light diode can be arranged at the light exit surface for illumination. Alternatively, the illumination can also be effected via fiber optics which transmit light from the proximal to the distal end of the endoscope. A suitable illumination coupling or light source, via which light is led into the fiber optics, can be arranged at the proximal end of the endoscope, said fiber optics then transmitting the light to the distal end to the mentioned light exit surface.

Particularly preferably, fiber optics which serve for the illumination are arranged in a cross-sectional region of the endoscope which is not filled out by endoscope optics, the first fluid channel and a working channel, wherein the fiber optics further preferably fill out the complete remaining free space in the inside of the endoscope in a manner surrounding endoscope optics, the first fluid channel and the working sleeve. Thus a maximal transmission of light through the endoscope shank can be achieved. Instead of fiber optics, one or more LEDs which serve for the illumination can be arranged in the region of the distal end-face of the endoscope which surrounds the viewing window, the exit opening of the first fluid channel and the opening of the working channel.

The invention is hereinafter described by way of example with reference to the attached figures. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a lateral view of the endoscope system according to the invention; and

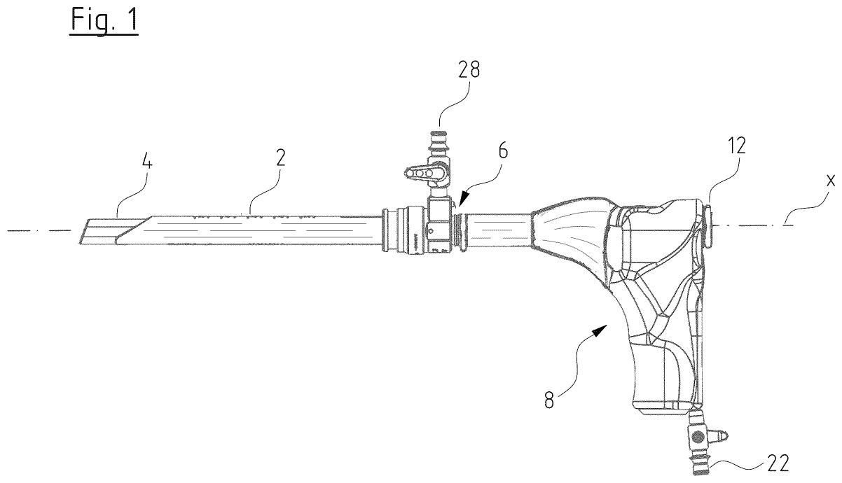

FIG. 2 is an enlarged plan view upon the distal end of the endoscope system according to the invention.

### DESCRIPTION OF PREFERRED EMBODIMENTS

Referring to the drawings, the endoscope system according to the invention as two essential elements comprises a working sleeve 2 and an endoscope 4 which can be inserted into this. The working sleeve 2 is configured in a tubular manner and the endoscope 4 is pushed through an opening or receiver 6 at the proximal end of the working sleeve 2 into the working sleeve 2, so that it exits out of the working sleeve 2 at the distal end. The endoscope 4 at its proximal end comprises a handle 8. A working channel 10 in the inside of the endoscope 4 is open to the proximal end on a receiver 12 through which a working insert or instrument can be pushed into the working channel 10.

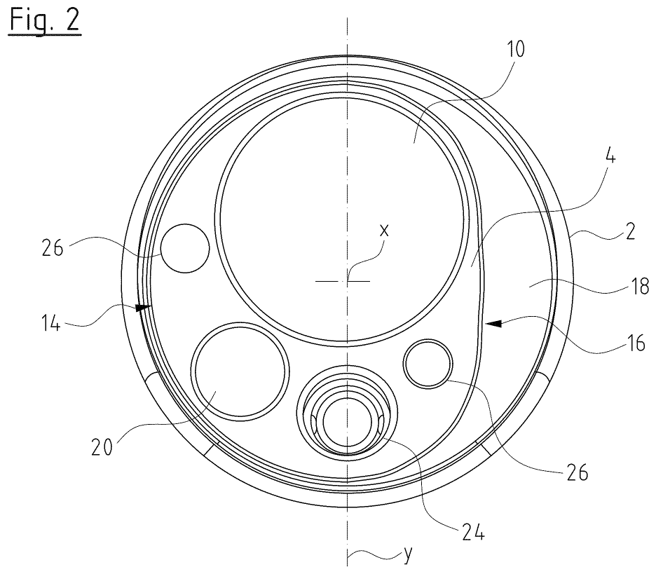

The working sleeve 2 comprises a circular cross section and in particular a circular inner cross section transverse to the longitudinal axis X which extends from the proximal to the distal end. The inner peripheral surface of the working sleeve 2 extends concentrically about the longitudinal axis X. The endoscope 4 has a cross section which in a first section is configured in a semicircular manner and in a second section in an oval manner. In this example, a first section of the outer periphery of the endoscope 4 extends over 180 degrees which is to say a first half, in FIG. 2 the half on the left of the axis Y. In this first section, the outer contour of the endoscope is semicircular and comprises a radius which corresponds essentially to the radius of the inner periphery of the working sleeve 2, so that the endoscope 4 with its outer periphery can slide along the inner peripheral surface 2 in the first section 14 when the endoscope 4 is rotated about the longitudinal or middle axis X of the working sleeve 2. This can be effected by way of a rotation in the opening 6 of the working sleeve 2. A second section 16 of the outer periphery of the endoscope 4, in FIG. 2 the section at the right of the axis Y, is configured in a flattened or oval manner, so that a free space 18 is formed between the outer periphery of the endoscope 4 and the inner periphery of the working sleeve 2. This free space 18 rotates together with the endoscope 4 on rotating the endoscope 4 in the inside of the working sleeve 2 about the longitudinal axis X. Seen in the diameter direction normal to the transverse axis Y which is shown in FIG. 2, herein a free space 18 exists only at one side of the longitudinal axis X, whereas the endoscope 4 with the outer periphery bears on the inner periphery of the working sleeve 2 at the side which is opposite or distanced in this diameter direction. As a whole, the endoscope 4 herewith with its outer peripheral surface bears on the inner periphery of the working sleeve 2 over a continuous section of roughly 180 degrees. By way of this, a large stability of the instrument also with regard to a buckling is achieved, since the endoscope 4 in the inside of the working sleeve 2 stabilizes or stiffens this working sleeve.

A first fluid channel 20 which ends at a first fluid connection 22 at the proximal end or handle 8 of the endoscope 4 is formed in the inside of the endoscope 4. Furthermore, the working channel 10 which extends in the proximal direction to the receiver 12 is open to the distal end of the endoscope 4. Furthermore, a viewing window 24 which can be configured for example as an objective lens is situated at the distal end of the endoscope 4, said viewing window forming the distal end of endoscope optics which extend through the endoscope shank 4 to the proximal end which is to say towards the handle 8, in the longitudinal direction X. An eyepiece can be provided on the handle 8 or a camera attached for recording images. Alternatively, a video chip could also be arranged in the viewing window 24 or behind the viewing window 24.

Two LEDs 26 are arranged at the distal end of the endoscope 4 for illumination. Alternatively to the LEDs 26, fiber optics which extend from the distal end to an illumination coupling on the handle 8 can also be arranged in the inside of the endoscope 4. The fiber optics could preferably fill out the complete free space which surrounds the working channel 10, the first fluid channel 20 and the endoscope optics.

In the shown example, the working channel 10 and the viewing window 24 are situated in the endoscope 4 such that their diameter axes lie on a common diameter axis Y transverse to the longitudinal axis X, which means extend along a common straight line. The first fluid channel 20 is arranged relative to the free space 18 which forms a second fluid channel, such that the first fluid channel 20 and the free space 18 are situated at opposite sides of the axis Y which is to say at sides of the viewing window 24 which are away from one another. The free space 18 as the second fluid channel is connected at the proximal end of the working sleeve 2 to a second fluid connection 28. The first fluid connection 22 and the second fluid connection 28 could be connected to hoses for the feed and discharge of fluid. The first fluid channel 20 serves as the feed, whereas the second fluid channel 18 as a discharge or return flow. The cross section of the free space 18 is larger than the cross section of the first fluid channel 20 and it is therefore ensured that a larger flow cross section is available for the return flow, so that a pressure increase due to the feed of fluid in the operational field can be avoided. It is ensured that the return flow is always larger than the feed flow. Since the opening of the first fluid channel 20 at the distal end and the opening of the free space 18 are situated on sides of the viewing window 24 which are away from one another, which is to say sides of the axis Y which are away from one another, it is ensured that a fluid flow which exits from the first fluid channel 20 and flows to the free space 18 flows over the viewing window 24 at the outer side and continuously rinses the field of view in the viewing direction and keeps it free of disturbing contaminations such as blood or tissue parts. This flow direction co-rotates on rotating the endoscope 4 in the working sleeve 2, so that independently of the angular position of the endoscope 4 in the working sleeve 2, a flow over the viewing window 24 is always ensured. In the present example, the opening of the first fluid channel 20 is offset somewhat towards the working channel 10 in a direction parallel to the axis Y, with respect to the viewing window 24. By way of this, it is rendered possible to optimally utilize the cross-sectional area of the endoscope 4. Furthermore, in the present example the viewing direction out of the viewing window 24 is angled, i.e. directed towards the working channel 10. On account of the offset arrangement of the opening of the fluid channel 20 one herewith succeeds in the fluid flow from the first fluid channel 20 to the free space 18 running through the field of view in the angled viewing direction in front of the viewing window 24.

The combination of a circular working sleeve 2 with a cross section of the endoscope 4 5 which is configured in an oval manner in a section, on the one hand permits the free rotatability of the endoscope 4 in the inside of the working sleeve 2 and on the other hand a large second fluid channel for the return of the fluid. At the same time, the endoscope cross section is optimally utilized for the working channel 10, the endoscope optics which end at the viewing window 24 and the fluid channel 20.

While specific embodiments of the invention have been shown and described in detail to illustrate the application of the principles of the invention, it will be understood that the invention may be embodied otherwise without departing from such principles.

### LIST OF REFERENCE CHARACTERS

2 working sleeve
4 endoscope
6 opening
8 handle
10 working channel
12 receiver
14 first section of the outer periphery
16 second section of the outer periphery
18 free space/second fluid channel
20 first fluid channel
22 first fluid connection
24 viewing window/distal end of the endoscope optics
26 LED
28 second fluid connection
X longitudinal axis, rotation axis
Y transverse axis

1. An endoscope system comprising:
a working sleeve; and
an endoscope configured to be inserted into the working sleeve, wherein a first fluid channel which is open to the distal end is formed in the inside of the endoscope,
wherein an outer cross section of the endoscope and an inner cross section of the working sleeve are matched to one another such that with the endoscope inserted into the working sleeve the endoscope in a first section of an outer periphery of the endoscope bears on an inner periphery of the working sleeve and in a second section it's the outer periphery of the endoscope is distanced to the inner periphery of the working sleeve,
wherein a free space, which extends over the axial length of the working sleeve and which forms a second fluid channel which is open to the distal end, is formed in a region between the inner periphery of the working sleeve and the second section of the outer periphery of the endoscope.

2. An endoscope system according to claim 1, wherein the endoscope, at it's the distal end of the endoscope, comprises a viewing window.

3. An endoscope system according to claim 2, wherein the opening of the first fluid channel and the free space which forms the second fluid channel are situated on sides of the viewing window which are away from one another.

4. An endoscope system according to claim 1, wherein at the distal end, the opening of the first fluid channel and the free space which defines the second fluid channel are arranged relative to a viewing window such that a fluid flow through a viewing field in front of the viewing window runs between the first and the second fluid channel.

5. An endoscope system according to claim 1, wherein the working sleeve has a circular inner cross section.

6. An endoscope system according to claim 1, wherein the endoscope in the first section of the outer periphery of the endoscope has an outer contour which is arc-shaped in cross section.

7. An endoscope system according to claim 1, wherein the outer contour of the endoscope is flattened or is configured in an oval arc shape, in the region of the second section of the outer periphery.

8. An endoscope system according to claim 1, wherein the endoscope is rotatable about a longitudinal axis X in the inside of the working sleeve.

9. An endoscope system according to claim 1, wherein the second fluid channel has a larger cross-sectional area than the first fluid channel.

10. An endoscope system according to claim 1, wherein the first fluid channel is configured as a feed channel and the second fluid channel is configured as a discharge channel.

11. An endoscope system according to claim 1, wherein the endoscope in the inside of the endoscope comprises a working channel which is open to the distal end.

12. An endoscope system according to claim 11, wherein the endoscope, the distal end of the endoscope, comprises a viewing window and wherein a cross-sectional axis of the working channel and a cross-sectional axis of the viewing window lie on a straight line that runs parallel to or along the diameter of the working sleeve.

13. An endoscope system according to claim 12, wherein the straight line which is defined by the cross section axes and the outer contour in the first section of the outer periphery of the endoscope define a semicircle.

14. An endoscope system according to claim 11, wherein the sum of the diameter of the viewing window and the working channel is larger than 75% of the inner diameter of the working sleeve.

15. An endoscope system according to claim 1, wherein the endoscope comprises endoscope optics which extend from the distal to the proximal end.

16. An endoscope system according to claim 1, wherein the endoscope at the distal end of the endoscope comprises at least one light exit surface.

17. An endoscope system according to claim 15, wherein fiber optics, which serve for illumination are arranged in a cross-sectional region of the endoscope which is not filled out by endoscope optics, the first fluid channel and a working channel or that one or more LEDs which serve for the illumination are arranged at the distal end of the endoscope.

18. An endoscope system according to claim 2, wherein the viewing window has a circular cross section.

19. An endoscope system according to claim 6, wherein the arc-shaped outer contour has a radius of curvature which corresponds essentially to the radius of the inner periphery of the working sleeve.

20. An endoscope system according to claim 11, wherein the working channel has a circular cross section.
